(19) United States
(12) Patent Application Publication
(10) Pub. No.: US 2002/0058311 A1
(43) Pub. Date: May 16, 2002

(54) Chimeric leptin fused to immunoglobulin domain and use

(76) Inventors: Michael Jospeh Browne, Welwyn Garden City (GB); Conrad Gerald Chapman, Orpington (GB); Helen Elizabeth Clinkenbeard, Hertford (GB); Jeffery Hugh Robinson, Epsom (GB)

Correspondence Address: GLAXOSMITHKLINE, Corporate Intellectual Property - UW2220, P.O. Box 1539, King of Prussia, PA, 19406-0939, US

(21) Appl. No.: 09/859,361

(22) Filed: May 17, 2001

Related U.S. Application Data
(63) Continuation of application No. 08/981,783, filed on Sep. 18, 1998, now abandoned.
Application No. 08/981,783 is a 371 of international application No. PCT/GB96/01388, filed on Jun. 11, 1996.

(30) Foreign Application Priority Data
Jun. 13, 1995 (GB) 9511935.0

(51) Int. Cl. C12P 21/04; A61K 39/395; C07K 16/18

(52) U.S. Cl. 435/69.7; 424/178.1; 530/391.1

(57) ABSTRACT

Chimeric leptin which are proteins comprising leptin or a mutant or a variant thereof fused to a human immunogobulin domain. One favoured immunoglobulin domain is the human immunoglobulin Fc domain. The chimeric derivatives of leptin have, despite their large molecular size, good pharmacological activity combined with prolonged clearance rates. These derivatives of leptin are therefore indicated to be particularly useful for the treatment or prophylaxis of obesity or diseases and conditions associated with obesity such as atherosclerosis, hypertension and type II diabetes.

The present invention relates to a novel compound being a novel chimeric protein, to a process for the preparation of such a compound, a pharmaceutical composition comprising such a compound and the use of such a compound in medicine, especially for the treatment of obesity and associated diseases.

European Patent Application, Publication number 0 464 533 discloses fusion proteins comprising various portions of the constant region of immunoglobulin molecules together with another human protein or part thereof. European Patent Application, Publication number 0 297 882 discloses fusion proteins comprising various portions of the plasminogen molecule with part of another human protein.

Zhang et al. (Nature: 372, 425 - 432; 1994) describe the positional cloning of a mouse obese gene and its human homologue. The sequence of the Open Reading Frame (ORF) of the mouse gene predicts a polypeptide of 167 amino acids and Zhang et al. predicted the presence of a signal sequence which would lead to the production of a mature protein of 146 residues. The human homologue was disclosed as having a similar size of 146 amino acids for the mature protein. Zhang et al. showed the presence of a primary translation product of approximate size of 18 kilodaltons (kD) with truncation to a 16 kD product on addition of microsomal membranes, consistent with the production of a pre-protein and the removal of an N-terminal signal sequence. Zhang et al also disclose the potential use of the human obese gene product (hereinafter ‘leptin’) in the treatment of obesity.

For effective, practical treatment of obesity a particularly desirable property of an obesity agent is a clearance rate in humans commensurate with patient acceptable treatment regimens, especially regimens for injectable therapies. Zhang et al. do not disclose information relating to the clearance rate of the active molecule in either mouse or humans.

The precise mechanism of action of leptin is currently unknown, however it is considered that in order to provide the observed pharmacological effects, leptin must interact with one or more receptors in the brain.

We have now discovered certain chimeric derivatives of leptin which surprisingly, despite their large molecular size, have good pharmacological activity combined with prolonged clearance rates. These chimeric derivatives of leptin are therefore indicated to be particularly useful for the treatment or prophylaxis of obesity and for the treatment or prophylaxis of diseases and conditions associated with obesity, such as atherosclerosis, hypertension and, especially, Type II diabetes. In particular these compounds are considered to be useful for administration by injection.

These compounds are also considered to be useful in cosmetic treatments for the improvement of body appearance.

Accordingly, the invention provides a chimeric leptin or a chimeric mutant or derivative of leptin.

One particular chimeric leptin is a protein comprising leptin or a mutant or variant thereof fused to a human immunoglobulin domain or a mutant or variant thereof.

Suitably, the chimeric protein comprises one human immunoglobulin domain.

Favourably , the human immunoglobulin domain is fused to the C-terminus of leptin.

One favoured human immunoglobulin is an human immunoglobulin Fc domain.

An example of a human immunoglobulin Fc domain is an IgG4PE variant in particular IgG4 hinge-CH₂-CH_{3.}PE. Other examples are IgG4, IgG1 and IgG1GT, in particular the hinge-CH₂-CH₃ region in each case.

The term “mutant or variant” used with respect to a particular protein encompasses any molecule such as a truncated or other derivative of the relevant protein which retains substantially the same activity in humans as the relevant protein. Such other derivatives can be prepared by the addition, deletion, substitution, or rearrangement of amino acids or by chemical modifications thereof.

The immunoglobulin may be of any subclass (IgG, IgM, IgA, IgE), but is preferably IgG, such as IgG1, IgG3 or IgG4. The said constant domain(s) or fragment thereof may be derived from the heavy or light chain or both. The invention encompasses mutations in the immunoglobulin component which eliminate undesirable properties of the native immunoglobulin, such as Fc receptor binding and/or introduce desirable properties such as stability. For example, Angal S., King D. J., Bodmer M. W., Turner A., Lawson A.D.G., Roberts G., Pedley B. and Adair R., Molecular Immunology vol30pp105-108, 1993, describe an IgG4 molecule where residue 241 (Kabat numbering) is altered from serine to proline. This change increases the serum half-life of the IgG4 molecule. Canfield S. M. and Morrison S. L., Journal of Experimental Medicine vol173pp1483-1491, describe the alteration of residue 248 (Kabat numbering) from leucine to glutamate in IgG3 and from glutamate to leucine in mouse IgG2b. Substitution of leucine for glutamate in the former decreases the affinity of the immunoglobulin molecule concerned for the FcγRI receptor, and substitution of glutamate for leucine in the latter increases the affinity. EP0307434 discloses various mutations including an L to E mutation at residue 248 (Kabat numbering) in IgG.

The constant domain(s) or fragment thereof is preferably the whole or a substantial part of the constant region of the heavy chain of human IgG. The IgG component suitably comprises the CH2 and CH3 domains and the hinge region including cysteine residues contributing to inter-heavy chain disulphide bonding.

For example when the IgG component is derived from IgG4 it includes cysteine residues 8 and 11 of the IgG4 hinge region (Pinck J. R. and Milstein C., Nature vol216pp941-942, 1967). Preferably the IgG4 component consists of amino acids corresponding to residues 1-12 of the hinge, 1-110 of CH2 and 1-107 of CH3 of IgG4 described by Ellison J., Buxbaum J. and Hood L., DNA vol1pp11-18, 1981. In one example of a suitable mutation in IgG4, residue 10 of the hinge (residue 241, Kabat numbering) is altered from serine (S) in the wild type to proline (P) and residue 5 of CH2 (residue 248, Kabat numbering) is altered from leucine (L) in the wild type to glutamate (E).

DNA polymers which encode mutants or variants of the human immunoglobulin may be prepared by site-directed mutagenesis of the cDNA which codes for the required protein by conventional methods such as those described by G. Winter et al in Nature 1982, 299, 756-758 or by Zoller and Smith 1982; Nucl. Acids Res., 10, 6487-6500, or deletion mutagenesis such as described by Chan and Smith in Nucl. Acids Res., 1984, 12, 2407-2419 or by G. Winter et al in Biochem. Soc. Trans., 1984; 12, 224-225 or polymerase chain reaction such as described by Mikaelian and Sergeant in Nucleic Acids Research, 1992, 20, 376.

When used herein ‘compound of the invention’ or ‘compounds of the invention’ relates to the above mentioned chimera.

In a further aspect, the invention provides a process for preparing a compound according to the invention which process comprises expressing DNA encoding said compound in a recombinant host cell and recovering the product.

The DNA polymer comprising a nucleotide sequence that encodes the compound also forms part of the invention.

The process of the invention may be performed by conventional recombinant techniques such as described in Maniatis et. al., Molecular Cloning - A Laboratory Manual; Cold Spring Harbor, 1982 and DNA Cloning vols I, II and III (D. M. Glover ed., IRL Press Ltd).

In particular, the process may comprise the steps of:

i) preparing a replicable expression vector capable, in a host cell, of expressing a DNA polymer comprising a nucleotide sequence that encodes said compound;

ii) transforming a host cell with said vector;

iii) culturing said transformed host cell under conditions permitting expression of said DNA polymer to produce said compound; and

iv) recovering said compound.

The invention also provides a process for preparing the DNA polymer by the condensation of appropriate mono-, di- or oligomeric nucleotide units.

The preparation may be carried out chemically, enzymatically, or by a combination of the two methods, in vitro or in vivo as appropriate. Thus, the DNA polymer may be prepared by the enzymatic ligation of appropriate DNA fragments, by conventional methods such as those described by D. M. Roberts et al in Biochemistry 1985, 24, 5090-5098.

The DNA fragments may be obtained by digestion of DNA containing the required sequences of nucleotides with appropriate restriction enzymes, by chemical synthesis, by enzymatic polymerisation on DNA or RNA templates, or by a combination of these methods.

Digestion with restriction enzymes may be performed in an appropriate buffer at a temperature of 20°-70° C., generally in a volume of 50 μl or less with 0.1- 10 μg DNA.

Enzymatic polymerisation of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10°-37° C., generally in a volume of 50 μl or less.

Enzymatic ligation of DNA fragments may be carried out using a DNA ligase such as T4 DNA ligase in an appropriate buffer at a temperature of 4° C. to ambient, generally in a volume of 50 μl or less.

The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments - A Laboratory Manual’ (ed. H. G. Gassen and A. Lang), Verlag Chemie, Weinheim (1982),or in other scientific publications, for example M. J. Gait, H.W.D. Matthes, M. Singh, B. S. Sproat, and R. C. Titrmas, Nucleic Acids Research, 1982, 10, 6243; B. S. Sproat and W. Bannwarth, Tetrahedron Letters, 1983, 24, 5771; M. D. Matteucci and M. H Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society,1983, 105, 661; N. D. Sinha, J. Biernat, J. McMannus, and H. Koester, Nucleic Acids Research, 1984, 12, 4539; and H.W.D. Matthes et al., EMBO Journal, 1984, 3, 801. Preferably an automated DNA synthesizer is employed.

The DNA polymer is preferably prepared by ligating two or more DNA molecules which together comprise a DNA sequence encoding the compound. A particular process in accordance with the invention comprises ligating a first DNA molecule encoding a said leptin or variant and a second DNA molecule encoding a said immunoglobulin domain or fragment thereof.

The DNA molecules may be obtained by the digestion with suitable restriction enzymes of vectors carrying the required coding sequences or by use of polymerase chain reaction technology.

The precise structure of the DNA molecules and the way in which they are obtained depends upon the structure of the desired product. The design of a suitable strategy for the construction of the DNA molecule coding for the compound is a routine matter for the skilled worker in the art.

The expression of the DNA polymer encoding the compound in a recombinant host cell may be carried out by means of a replicable expression vector capable, in the host cell, of expressing the DNA polymer. The expression vector is novel and also forms part of the invention.

The replicable expression vector may be prepared in accordance with the invention, by cleaving a vector compatible with the host cell to provide a linear DNA segment having an intact replicon, and combining said linear segment with one or more DNA molecules which, together with said linear segment, encode the compound, under ligating conditions.

The ligation of the linear segment and more than one DNA molecule may be carried out simultaneously or sequentially as desired.

Thus, the DNA polymer may be preformed or formed during the construction of the vector, as desired.

The choice of vector will be determined in part by the host cell, which may be prokaryotic, such as E. coli, or eukaryotic, such as mouse C127, mouse myeloma, chinese hamster ovary, Cos1 or Hela cells, fungi e.g. filamentous fungi or unicellular yeast or an insect cell such as Drosophila. The host cell may also be a transgenic animal.

A preferred host cell is Cos1.

Suitable vectors include plasmids, bacteriophages, cosmids and recombinant viruses derived from, for example, baculoviruses, vaccinia or Semliki Forest virus.

The preparation of the replicable expression vector may be carried out conventionally with appropriate enzymes for restriction, polymerisation and ligation of the DNA, by procedures described in, for example, Maniatist et. al., cited above. Polymerisation and ligation may be performed as described above for the preparation of the DNA polymer. Digestion with restriction enzymes may be performed in an appropriate buffer at a temperature of 20°-70° C., generally in a volume of 50 μl or less with 0.1-10 μg DNA.

The recombinant host cell is prepared, in accordance with the invention, by transforming a host cell with a replicable expression vector of the invention under transforming conditions. Suitable transforming conditions are conventional and are described in, for example, Maniatis et al., cited above, or “DNA Cloning” Vol. II, D. M. Glover ed., IRL Press Ltd, 1985.

The choice of transforming conditions is determined by the host cell. Thus, a bacterial host such as E. coli may be treated with a solution of CaCl₂ (Cohen et al, Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol. Mammalian cells in culture may be transformed by calcium co-precipitation of the vector DNA onto the cells.

The invention also extends to a host cell transformed or transfected with a replicable expression vector of the invention.

Culturing the transformed host cell under conditions permitting expression of the DNA polymer is carried out conventionally, as described in, for example, Maniatis et al and “DNA Cloning” cited above. Thus, preferably the cell is supplied with nutrient and cultured at a temperature below 45° C.

The expression product is recovered by conventional methods according to the host cell. Thus, where the host cell is bacterial, such as E. coli it may be lysed physically, chemically or enzymatically and the protein product isolated from the resulting lysate. If the product is to be secreted from the bacterial cell it may be recovered from the periplasmic space or the nutrient medium. Where the host cell is mammalian, the product may generally be isolated from the nutrient medium.

The DNA polymer may be assembled into vectors designed for isolation of stable transformed mammalian cell lines expressing the product; e.g. bovine papillomavirus vectors or amplified vectors in chinese hamster ovary cells (DNA cloning Vol.II D. M. Glover ed. IRL Press 1985; Kaufman, R. J. et. al., Molecular and Cellular Biology 5, 1750-1759, 1985; Pavlakis G. N. and Hamer, D. H., Proceedings of the National Academy of Sciences (USA) 80, 397-401, 1983; Goeddel, D. V. et al., European Patent Application No.0093619, 1983).

The activity of the chimeric leptin is determined by injecting it intraperitoneally, intravenously or subcutaneously into test animals such as rodents, for example mice or rats, or primates, for example rhesus monkeys. In order to maximise activity, the test animals are preferably overweight or obese animals that have been made overweight by feeding them on a high fat or other palatable diet, or have acquired fat through the ageing process. In the case of mice, however, the ideal strain is the genetically obese (ob/ob) mouse. The effect of the active compound is seen as a reduction in food intake or increase in metabolic rate or oxygen consumption. Multiple injections of the active compound—at most twice daily—over a period of a week for rodents or a month for primates, also cause a reduction in body weight and in the size of discrete adipose tissue depots.

Clearance rates are determined by conventional plasma assay using ob-antibodies, for example ELISA methodology.

As indicated above the compounds of the present invention have useful pharmaceutical properties, in particular anti obesity activity and also for the treatment of diseases associated with obesity, such as atherosclerosis, hypertension and, especially, Type II diabetes.

In use the compound will normally be employed in the form of a pharmaceutical composition in association with a human pharmaceutical carrier, diluent and/or excipient, although the exact form of the composition will depend on the mode of administration.

The active compound may be formulated for administration by any suitable route and is preferably in unit dosage form. Advantageously, the composition is suitable for oral, rectal, topical, parenteral, intravenous or intramuscular administration or through the respiratory tract Preparations may be designed to give slow release of the active ingredient.

The compositions of the invention may be in the form of tablets, capsules, sachets, vials, powders, granules, lozenges, suppositories, reconstitutable powders, or liquid preparations such as oral or sterile parenteral solutions or suspensions. Topical formulations are also envisaged where appropriate.

The invention therefore further provides a pharmaceutical composition comprising a compound of the invention and a pharmaceutically acceptable carrier. The dosage ranges for administration of the compounds of the present invention are those to produce the desired therapeutic effect. Dosage will generally vary with age, extent or severity of the medical condition and contraindications, if any. For example in the treatment of obsity the unit dosage can vary from less than 1 mg to 300 mg, but typically will be in the region of 1 to 20 mg per dose, in one or more doses, such as one to six doses per day, such that the daily dosage is in the range 0.02-40 mg/kg.

Dosages and compositions for the treatment of diseases associated with obesity such as atherosclerosis, hypertension and, especially, Type II diabetes are selected from an equivalent range to that used in the treatment of obesity.

Compositions suitable for injection may be in the form of solutions, suspensions or emulsions, or dry powders which are dissolved or suspended in a suitable vehicle prior to use.

Fluid unit dosage forms are prepared utilising the compound and a pyrogen-free sterile vehicle. The compound, depending on the vehicle and concentration used, can be either dissolved or suspended in the vehicle. Solutions may be used for all forms of parenteral administration, and are particularly used for intravenous infection. In preparing solutions the compound can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and/or local anaesthetic agents may be dissolved in the vehicle.

Dry powders which are dissolved or suspended in a suitable vehicle prior to use may be prepared by filling pre-sterilised drug substance and other ingredients into a sterile container using aseptic technique in a sterile area. Alternatively the drug and other ingredients may be dissolved in an aqueous vehicle, the solution is sterilised by filtration and distributed into suitable containers using aseptic technique in a sterile area. The product is then freeze dried and the containers are sealed aseptically.

Parenteral suspensions, suitable for intramuscular, subcutaneous or intraderrnal injection, are prepared in substantially the same manner, except that the sterile compound is suspended in the sterile vehicle, instead of being dissolved and sterilisation cannot be accomplished by filtration. The compound may be isolated in a sterile state or alternatively it may be sterilised after isolation, e.g. by gamma irradiation. Advantageously, a suspending agent for example polyvinylpyrrolidone is included in the composition to facilitate uniform distribution of the compound.

Compositions suitable for administration via the respiratory tract include aerosols, nebulisable solutions or microfine powders for insufflation. In the latter case, particle size of less than 50 microns, especially less than 10 microns, is preferred. Such compositions may be made up in a conventional manner and employed in conjunction with conventional administration devices.

In a further aspect there is provided a method of treating obesity or diseases associated with obesity, such as atherosclerosis, hypertension and, especially, Type II diabetes, in human or non-human mammals which comprises administering to the sufferer an effective, non-toxic amount of a compound of the invention.

Suitable non-human mammals are domestic mammals such as dogs and cats.

The invention further provides a compound of the invention for use as an active therapeutic substance, in particular for use in treating obesity or diseases associated with obesity, such as atherosclerosis, hypertension and, especially, Type II diabetes.

The invention also provides the use of a compound of the invention in the manufacture of a medicament for treating obesity or diseases associated with obesity, such as atherosclerosis, hypertension and, especially, Type II diabetes.

As indicated above the invention also encompasses cosmetic treatments.

Accordingly, there is also provided a compound of the invention for use in the cosmetic treatment of human or non-human mammals.

There is also provided a method for the cosmetic treatment of a human or non-human mammal, which treatment comprises administering an effective, non-toxic amount of a compound of the invention to a human or non-human mammal in need thereof.

Cosmetic treatment suitably includes treatment for the improvement of body appearance, such as weight reduction treatment.

The invention also extends to a cosmetic composition, comprising a compound of the invention and a carrier therefor.

Compositions of the invention including cosmetic compositions are formulated using known methods, for example those described in standard text books of pharmaceutics and cosmetics, such as Harry's Cosmeticology published by Leonard Hill Books, Remington's Pharmaceutical Sciences, the British and US Pharmacopoeias.

No unexpected toxicological effects are expected when compounds of the invention are administered in accordance with the present invention.

The following Examples illustrate the invention but do not limit it in any way.

### EXAMPLE 1

Construction of DNA coding for fusion protein leptin 1-167/IgG4 hinge-CH2-CH3 The gene coding for a fusion protein comprising human leptin and the hinge-CH2-CH3 region of human IgG4 is created by recombinant DNA technology, preferably by a two-step recombinant PCR method.

The human ‘ob’ gene has been prepared synthetically based on the amino acid sequence of Zhang et al, and assembled in the pcDNA3 vector.

The cDNA encoding full length human leptin, nucleotides 1-501 is joined at the 3′ end to the 5′ end of the hinge-CH2-CH3 region of the cDNA coding for the human IgG4 protein, shown as nucleotides 502-1188 in the DNA sequence below. (Table 1.)

The encoded protein sequence of the leptin/IgG4 chimera is given in Table 2. Leptin 1-167 (numbering as Y. Zhang, R. Proenca, M. Maffei, M. Barone, L. Leopold & J. Friedman. Nature 372:425-432), and IgG4 hinge-CH2-CH3 168-396 (sequence as Kabat).

The fusion protein was expressed transiently in Cos1 cells using the pCDN vector system, as described in International Patent Application Publication number WO 96/04388. The mature protein was exported from the cells into the culture medium and was detected by anti-leptin antibody. It was shown to to have a size consistent with the predicted structure by Western blotting analysis under both reducing and nonreducing conditions.

TABLE 1
DNA sequence of ob IgG4 chimera, 1188bp

```
ATGCATTGGGGAACCCTGTGCGGATTCTTGTGGCTTTGGCCCTATCTTTTCTATGTCCAA
                                                                                                                    60
GCTGTGCCCATCCAAAAAGTCCAAGATGACACCAAAACCCTCATCAAGACAATTGTCACC
                                                                                                                  120
AGGATCAATGACATTTCACACACGCAGTCAGTCTCCTCCAAACAGAAAGTCACCGGTTTG
                                                                                                                  180
GACTTCATTCCTGGGCTCCACCCCATCCTGACCCTGTCCAAGATGGACCAGACACTGGCA
                                                                                                                  240
GTCTACCAACAGATCCTCACATCGATGCCTTCCAGAAACGTGATCCAAATATCCAACGAC
                                                                                                                  300
CTGGAGAACCTCCGGGATCTTCTTCACGTGCTGGCCTTCTCTAAGAGCTGCCACTTGCCC
                                                                                                                  360
TGGGCCAGTGGCCTGGAGACCTTGGACAGCCTGGGGGGTGTCCTCGAGGCTTCAGGCTAC
                                                                                                                  420
TCCACAGAGGTGGTGGCCCTGAGCAGGCTGCAGGGGTCTCTGCAGGACATGCTGTGGCAG
                                                                                                                  480
CTGGACCTCAGCCCCGGGTGCGAGTCCAAATATGGTCCCCCATGCCCATCATGCCCAGCA
                                                                                                                  540
CCTGAATTTCTGGGGGGACCATCAGTCTTCCTGTTCCCCCCAAAACCCAAGGACACTCTC
                                                                                                                  600
ATGATCTCCCGGACCCCTGAGGTCACGTGCGTGGTGGTGGACGTGAGCCAGGAAGACCCC
                                                                                                                  660
GAGGTCCAGTTCAACTGGTACGTGGATGGCGTGGAGGTGCATAATGCCAAGACAAAGCCG
                                                                                                                  720
CGGGAGGAGCAGTTCAACAGCACGTACCGTGTGGTCAGCGTCCTCACCGTCCTGCACCAG
                                                                                                                  780
GACTGGCTGAACGGCAAGGAGTACAAGTGCAAGGTCTCCAACAAAGGCCTCCCGTCATCG
                                                                                                                  840
ATCGAGAAAACCATCTCCAAAGCCAAAGGGCAGCCCCGAGAGCCACAGGTGTACACCCTG
                                                                                                                  900
CCCCCATCCCAGGAGGAGATGACCAAGAACCAGGTCAGCCTGACCTGCCTGGTCAAAGGC
                                                                                                                  960
TTCTACCCCAGCGACATCGCCGTGGAGTGGGAGAGCAATGGGCAGCCGGAGAACAACTAC
                                                                                                                1020
AAGACCACGCCTCCCGTGCTGGACTCCGACGGATCCTTCTTCCTCTACAGCAGGCTAACC
                                                                                                                1080
GTGGACAAGAGCAGGTGGCAGGAGGGGAATGTCTTCTCATGCTCCGTGATGCATGAGGCT
                                                                                                                1140
CTGCACAACCACTACACACAGAAGAGCCTCTCCCTGTCTCTGGGTAAA
                                                                                        1188
```

TABLE 2
Amino acid sequence of leptin/IgG4 chimera, 396aa

1 MHWGTLCGFL WLWPYLFYVQ AVPIQKVQDD TKTLIKTIVT
RINDISHTQS
51 VSSKQKVTGL DFIPGLHPIL TLSKMDQTLA VYQQILTSMP SRNVIQISND
101 LENLRDLLHV LAFSKSCHLP WASGLETLDS LGGVLEASGY
STEVVALSRL
151 QGSLQDMLWQ LDLSPGCESK YGPPCPSCPA PEFLGGPSVF
LFPPKPKDTL
201 MISRTPEVTC VVVDVSQEDP EVQFNWYVDG VEVHNAKTKP
REEQFNSTYR
251 VVSVLTVLHQ DWLNGKEYK CKVSNKGLPSS IEKTISKAKG
QPREPQVYTL
301 PPSQEEMTKN QVSLTCLVKG FYPSDIAVEW ESNGQPENNY
KTITPPVLDSD
351 GSFFLYSRLT VDKSRWQEGN VFSCSVMHEA LHNHYTQKSL
SLSLGK

### EXAMPLE 2

Construction of DNA coding for fusion protein ob 1-167/IgG4 hinge-CH2-CH3 PE variant

The gene coding for a fusion protein comprising the human ‘ob’ protein and the Hinge-CH2-CH3 region of human IgG4 PE (a form of IgG4 mutated as below) is created by recombinant DNA technology, preferably by a two-step recombinant PCR method.

The cDNA coding for the complete human leptin, amino acids 1-167(numbering as Y. Zhang, R. Proenca, M. Maffei, M. Barone, L. Leopold & J. Friedman. Nature 372: 425-432) is joined at the 3′ end to the 5′ end of the hinge-CH2-CH3 region of the cDNA coding for the human IgG4 (PE variant) protein, shown as amino acids 168-396 in the protein sequence below.

The human ‘ob’ gene has been prepared synthetically based on the amino acid sequence of Zhang et al, and assembled in the pcDNA3 vector. The encoded protein sequence is given in Table 2.

Human IgG4 heavy chain PE variant. In IgG4 PE, residue 10 of the hinge (residue 241, Kabat numbering) is altered from serine (S) in the wild type to proline (P) and residue 5 of CH2 (residue 248, Kabat numbering) is altered from leucine (L) in the wild type to glutamate (E). Angal S., King D. J., Bodmer M. W., Turner A., Lawson A.D.G., Roberts G., Pedley B. and Adair R., Molecular Immunology vol30pp105-108, 1993, describe an IgG4 molecule where residue 241 (Kabat numbering) is altered from serine to proline. This change increases the serum half-life of the IgG4 molecule.

The IgG4 PE variant was created using PCR mutagenesis on the synthetic human IgG4 heavy chain cDNA. The sequence of the IgG4 PE variant is described in Table 1. The residues of the IgG4 nucleotide sequence which were altered to make the PE variant are as follows:

referring to Table 1:

residue 322 has been altered to “C” in the PE variant from “T” in the wild type;

residue 333 has been altered to “G” in the PE variant from “A” in the wild type; and

residues 343-344 have been altered to “GA” in the PE variant from “CT” in the wild type.

The fusion protein was expressed transiently in Cos1 cells using the pCDN vector system, as described in International Patent Application Publication number WO 96/04388. The mature protein was exported from the cells into the culture medium and was detected by anti-leptin antibody. It was shown to to have a size consistent with the predicted structure by Western blotting analysis under both reducing and nonreducing conditions.

TABLE 3
DNA sequence of IgG4 PE variant, 984bp

```
SEQ ID No:1
GCTAGTACCAAGGGCCCATCCGTCTTCCCCCTGGCGCCCTGCTCCAGGAGCACCTCCGAG
                                                                                                                    60
AGCACgGCCGCCCTGGGCTGCCTCGTCAAGGACTACTTCCCCGAACCGGTGACGGTGTCG
                                                                                                                  120
TGGAACTCAGGCGCCCTGACCAGCGGCGTGCACACCTTCCCGGCTGTCCTACAGTCCTCA
                                                                                                                  180
GGACTCTACTCCCTCAGCAGCGTGGTGACCGTCCCCTCCAGCAGCTTGGGCACGAAGACC
                                                                                                                  240
TACACCTGCAACGTAGATCACAAGCCCAGCAACACCAAGGTGGACAAGAGAGTTGAGTCC
                                                                                                                  300
AAATATGGTCCCCCATGCCCAcGATGCCCAGCgCCTGAaTTtgaGGGGGGACCATCAGTC
                                                                                                                  360
TTCCTGTTCCCCCCAAAACCCAAGGACACTCTCATGATCTCCCGGACCCCTGAGGTCACG
                                                                                                                  420
TGCGTGGTGGTGGACGTGAGCCAGGAAGACCCCGAGGTGCAGTTCAACTGGTACGTGGAT
                                                                                                                  480
GGCGTGGAGGTGCATAATGCCAAGACAAAGCCGCGGGAGGAGCAGTTCAACAGCACGTAC
                                                                                                                  540
CGTGTGGTCAGCGTCCTCACCGTCCTGCACCAGGACTGGCTGAACGGCAAGGAGTACAAG
                                                                                                                  600
TGCAAGGTCTCCAACAAAGGCCTCCCGTCaTCgATCGAGAAAACCATCTCCAAAGCCAAA
                                                                                                                  660
GGGCAGCCCCGAGAGCCACAGGTGTACACCCTGCCCCCATCCCAGGAGGAGATGACCAAG
                                                                                                                  720
AACCAGGTCAGCCTCACCTGCCTGGTCAAAGGCTTCTACCCCAGCGACATCGCCGTGGAG
                                                                                                                  780
TGGGAGAGCAATGGGCAGCCGGAGAACAACTACAAGACCACGCCTCCCGTGCTGGACTCC
                                                                                                                  840
GACGGaTCCTTCTTCCTCTACAGCAGGCTAACCGTGGACAAGAGCAGCTGGCAGGAGGGG
                                                                                                                  900
AATGTCTTCTCATGCTCCGTGATGCATGAGGCTCTGCACAACCACTACACACAGAAGAGC
                                                                                                                  960
CTCTCCCTGTCTCTGGCTAAATGA
                                            984
```

TABLE 3A
DNA sequence of ob/IgG4PE chimera, 1188bp

```
ATGCATTGGGGAACCCTGTGCGGATTCTTCTGGCTTTGGCCCTATCTTTTCTATGTCCAA
                                                                                                                    60
GCTGTGCCCATCCAAAAAGTCCAAGATGACACCAAAACCCTCATCAAGACAATTGTCACC
                                                                                                                  120
AGGATCAATGACATTTCACACACGCAGTCAGTCTCCTCCAAACAGAAACTCACCGGTTTG
                                                                                                                  180
GACTTCATTCCTGGGCTCCACCCCATCCTGACCCTGTCCAAGATGGACCAGACACTGGCA
                                                                                                                  240
GTCTACCAACAGATCCTCACATCGATGCCTTCCAGAAACGTGATCCAAATATCCAACGAC
                                                                                                                  300
CTGGAGAACCTCCGGGATCTTCTTCACGTGCTGGCCTTCTCTAAGAGCTGCCACTTGCCC
                                                                                                                  360
TGGGCCAGTGGCCTGGAGACCTTGGACAGCCTGGGGGGTGTCCTCGAGGCTTCAGGCTAG
                                                                                                                  420
TCCACAGAGGTGGTGGCCCTGAGCAGGCTGCAGGGCTCTCTGCAGGACATGCTGTGGCAG
                                                                                                                  480
CTGGACCTCAGCCCCGGGTGCGAGTCCAAATATGGTCCCCCATGCCCAcCATGCCCAGCg
                                                                                                                  540
CCTGAATTTGAGGGGGGACCATCAGTCTTCCTGTTCCCCCCAAAACCCAAGGACACTCTC
                                                                                                                  600
ATGATCTCCCGGACCCCTGAGGTCACGTGCGTGGTGGTGGACGTGAGCCAGGAAGACCCC
                                                                                                                  660
GAGGTCCAGTTCAACTGGTACGTGGATGGCGTGGAGGTGCATAATGCCAAGACAAAGCCG
                                                                                                                  720
CGGGAGGAGCAGTTCAACAGCACGTACCGTGTGGTCAGCGTCCTCACCGTCCTGCACCAG
                                                                                                                  780
GACTGGCTGAACGGCAAGGAGTACAAGTGCAAGGTCTCCAACAAAGGCCTCCCGTCATCG
                                                                                                                  840
ATCGAGAAAACCATCTCCAAAGCCAAAGGGCAGCCCCGAGAGCCACAGGTGTACACCCTG
                                                                                                                  900
CCCCCATCCCAGGAGGAGATGACCAAGAACCAGGTCAGCCTGACCTGCCTGGTCAAAGGC
                                                                                                                  960
TTCTACCCCAGCGACATCGCCGTGGAGTTGGAGAGCAATGGGCAGCCGGAGAACAACTAC
                                                                                                                1020
AAGACCACGCCTCCCGTGCTGGACTCCGACGGATCCTTCTTCCTCTACAGCAGGCTAACC
                                                                                                                1080
GTGGACAAGACCAGGTGGCACGAGGGGAATGTCTTCTCATGCTCCGTGATGCATGAGGCT
                                                                                                                1140
CTGCACAACCACTACACACAGAAGAGCCTCTCCCTGTCTCTGGGTAAA
                                                                                          1188
```

TABLE 4
Amino acid sequence of ob 1-167/IgG4 hinge-CH2-CH3
PE variant chimera 396aa

SEQ ID No: 2

| | |
|---|---|
| 1 | MHWGTLCGFL WLWPYLFYVQ AVPIQKVQDD TKTLIKTIVT |
| | RINDISHTQS |
| 51 | VSSKQKVTGL DFIPGLHPIL TLSKMDQTLA VYQQILTSMP |
| | SRNVIQISND |
| 101 | LENLRDLLHV LAFSKSCHLP WASGLETLDS LGGVLEASGY |
| | STEVVALSRL |
| 151 | QGSLQDMLWQ LDLSPGCESK YGPPCPPCPA PEFEGGPSVF |
| | LFPPKPKDTL |
| 201 | MISRTPEVTC VVVDVSQEDP EVQFNWYVDG VEVHNAKTKP |
| | REEQFNSTYR |
| 251 | VVSVLTVLHQ DWLNGKEYKC KVSNKGLPSS IEKTISKAKG |
| | QPREPQVYTL |
| 301 | PPSQEEMTKN QVSLTCLVKG FYPSDIAVEW ESNGQPENNY |
| | KTTPPVLDSD |
| 351 | GSFFLYSRLT VDKSRWQEGN VFSCSVMHEA LHNHYTQKSL |
| | SLSLGK |

### EXAMPLE 3

Construction of DNA coding for fusion protein leptin 1-167/IgG1 hinge-CH2-CH3

The gene coding for a fusion protein comprising human leptin and the hinge-CH2-CH3 region of human IgG1 is created by recombinant DNA technology, preferably by a two-step recombinant PCR method.

The human ‘ob’ gene has been prepared synthetically based on the amino acid sequence of Zhang et al, and assembled in the pcDNA3 vector.

The cDNA encoding full length human leptin, nucleotides 1-501 is joined at the 3′ end to the 5′ end of the hinge-CH2-CH3 region of the cDNA coding for the human IgG1 protein, shown as nucleotides 502-1197 in the DNA sequence below. (Table 1.) The encoded protein sequence of the leptin/IgG1 chimera is given in Table 2. Leptin 1-167 (numbering as Y. Zhang, R. Proenca, M. Maffei, M. Barone, L. Leopold & J. Friedman. Nature 372: 425-432) and IgG1 hinge-CH2-CH3 shown as amino acids 168-399.

The gene coding for the human IgG1 contains a number of nucleotide substitutions compared to the IgG1 molecule described by Ellison J. W., Berson B. J. and Hood L. E., Nucleic Acids Research vol 10 No. 13 pp4071-4079, 1982. The IgG1 nucleotides which differ from the Ellison J. W. et al published sequence and the resulting amino acid substitutions are as follows (nucleotide numbering as in table 1)

nucleotide 513 is “G” in this variant compared to “T” in the Ellison et al sequence (silent mutation)

nucleotides 514-516 are “GCC” in this variant compared to “TGT” in the Ellison et al sequence (resulting in substitution of Ala for Cys in this variant, amino acid 172 in table 2)

nucleotide 759 is “T” in this variant compared to “G” in the Ellison et al sequence (silent mutation)

nucleotide 924 is “G” in this variant compared to “T” in the Ellison et al sequence (resulting in substitution of Glu for Asp in this variant, amino acid 308 in table2)

nucleotide 928 is “A” in this variant compared to “C” in the Ellison et al sequence (resulting in substitution of Met for Val in this variant, amino acid 310 in table 2)

nucleotide 1077 is “T” in this variant compared to “C” in the Ellison et al sequence (silent mutation)

nucleotide 1197 is “G” in this variant compared to “A” in the Ellison et al sequence (silent mutation)

The fusion protein was expressed transiently in Cos1 cells using the pCDN vector system, as described in International Patent Application Publication number WO 96/04388. The mature protein was exported from the cells into the culture medium and was detected by anti-leptin antibody. It was shown to to have a size consistent with the predicted structure by Western blotting analysis under both reducing and nonreducing conditions.

TABLE 5
DNA sequence of ob/IgG1 chimera 1197bp

```
ATGCATTGGGGAACCCTGTGCGGATTCTTGTGGCTTTGGCCCTATCTTTTCTATGTCCAA
                                                                                                                    60
GCTGTGCCCATCCAAAAAGTCCAAGATGACACCAAAACCCTCATCAAGACAATTGTCACC
                                                                                                                  120
AGGATCAATGACATTTCACACACGCAGTCAGTCTCCTCCAAACAGAAAGTCACCGGTTTG
                                                                                                                  180
GACTTCATTCCTGGGCTCCACCCCATCCTGACCCTGTCCAAGATGGACCAGACACTGGCA
                                                                                                                  240
GTCTACCAACAGATCCTCACATCGATGCCTTCCAGAAACGTGATCCAAATATCCAACGAC
                                                                                                                  300
CTGGAGAACCTCCGGGATCTTCTTCACGTGCTGGCCTTCTCTAAGAGCTGCCACTTGCCC
                                                                                                                  360
TGGGCCAGTGGCCTGGAGACCTTGGACAGCCTGGGGGGTGTCCTCGAGGCTTCAGGCTAC
                                                                                                                  420
TCCACAGAGGTGGTGGCCCTGAGCAGGCTGCAGGGGTCTCTGCAGGACATGCTGTGGCAG
                                                                                                                  480
CTGGACCTCAGCCCCGGGTGCGAGCCCAAATCGGCCGACAAAACTCACACATGCCCACCG
                                                                                                                  540
TGCCCAGCACCTGAACTCCTGGGGGGACCGTCAGTCTTCCTCTTCCCCCCAAAACCCAAG
                                                                                                                  600
GACACCCTCATGATCTCCCCGACCCCTGAGGTCACATGCGTGGTGGTGGACGTGAGCCAC
                                                                                                                  660
GAAGACCCTGAGGTCAAGTTCAACTGGTACGTGGACGGCGTGGAGGTGCATAATGCCAAG
                                                                                                                  720
ACAAAGCCGCGGGAGGAGCAGTACAACAGCACGTACCGTGTGGTCAGCGTCCTCACCGTC
                                                                                                                  780
CTGCACCAGGACTGGCTGAATGGCAAGGAGTACAAGTGCAAGGTCTCCAACAAAGCCCTC
                                                                                                                  840
CCAGCCCCCATCGAGAAAACCATCTCCAAAGCCAAAGGGCAGCCCCGAGAACCACAGGTG
                                                                                                                  900
TACACCCTGCCCCCATCCCGGGAGGAGATGACCAAGAACCAGGTCAGCCTGACCTGCCG
                                                                                                                  960
GTCAAAGGCTTCTATCCCAGCGACATCGCCGTGGAGTGGGAGAGCAATGGGCAGCCGGAG
                                                                                                                1020
AACAACTACAAGACCACGCCTCCCGTCCTGGACTCCGACGGCTCCTTCTTCCTCTATAGC
                                                                                                                1080
AAGCTCACCGTGGACAAGAGCAGGTGGCAGCAGGGGAACGTCTTCTCATGCTCCGTGATG
                                                                                                                1140
CATGAGGCTCTGCACAACCACTACACGCAGAAGAGCCTCTCCCTGTCTCCGGGTAAG
                                                                                                          1197
```

TABLE 6
Amino acid sequence of leptin/IgG1 chimera, 399aa

| | |
|---|---|
| 1 | MHWGTLCGFL WLWPYLFYVQ AVPIQKVQDD TKTLIKTIVT |
| | RINDISHTQS |
| 51 | VSSKQKVTGL DFIPGLHPIL TLSKMDQTLA VYQQILTSMP |
| | SRNYIQISND |
| 101 | LENLRDLLHV LAFSKSCHLP WASGLETLDS LGGVLEASGY |
| | STEVVALSRL |
| 151 | QGSLQDMLWQ LDLSPGCEPK SADKTHTCPP CPAPELLGGP |
| | SVFLFPPKPK |
| 201 | DTLMISRTPE VTCVVVDVSH EDPEVKFNWY VDGVEVHNAK |
| | TKPREEQYNS |
| 251 | TYRVVSVLTV LHQDWLNGKB YKCKVSNKAL PAPIEKTISK |
| | AKGQPREPQV |
| 301 | YTLPPSREEM TKNQVSLTCL VKGFYPSDIA VEWESNGQPE |
| | NNYKTTPPVL |
| 351 | DSDGSFFLYS KLTVDKSRWQ QGNVFSCSVM HEALHNHYTQ |
| | KSLSLSPGK |

### EXAMPLE 4

Construction of DNA coding for fusion protein leptin 1-167/IgG1 hinge-CH2-CH3 GT linker variant

The gene coding for a fusion protein comprising human leptin and the hinge-CH2-CH3 region of human IgG1 with a ‘GT’ two amino acid linker between the two parts of the fusion molecule, is created by recombinant DNA technology, preferably by a two-step recombinant PCR method.

The human ‘ob’ gene has been prepared synthetically based on the amino acid sequence of Zhang et al, and assembled in the pcDNA3 vector.

The cDNA encoding the full length human leptin (nucleotides 1-501) is joined at the 3′ end to the 5′ end of the hinge-CH2-CH3 region of the IgG1 cDNA (nucleotides 508-1203). The two amino acid linker between the two parts of the fusion is encoded by the nucleotide sequence GGTACC (502-507). See Table 1.

The encoded protein sequence of the leptin/IgG1 (GT) chimera is given in Table 2. Leptin 1-1 (numbering as Y. Zhang, R. Proenca, M. Maffei, M. Barone, L. Leopold & J. Friedman. Nature 372:425-432), followed by the GT linker (168-169) and IgG1 H-CH2-CH3 170-401.

The gene coding for the human IgG1 contains a number of nucleotide substitutions compared to the IgG1 molecule described by Ellison J. W., Berson B. J. and Hood L. E., Nucleic Acids Research vol 10 No. 13 pp4071-4079, 1982. The IgG1 nucleotides which differ from the Ellison J. W. et al published sequence and the resulting amino acid substitutions are as follows (nucleotide numbering as in table 1)

nucleotide 519 is “G” in this variant compared to “T” in the Ellison et al sequence (silent mutation)

nucleotides 520-522 are “GCC” in this variant compared to “TGT” in the Ellison et al sequence (resulting in substitution of Ala for Cys in this variant, amino acid 174 in table 2)

nucleotide 759 is “T” in this variant compared to “G” in the Ellison et al sequence (silent mutation)

nucleotide 924 is “G” in this variant compared to “T” in the Ellison et al sequence (resulting in substitution of Glu for Asp in this variant, amino acid 308 in table2)

nucleotide 928 is “A” in this variant compared to “C” in the Ellison et al sequence (resulting in substitution of Met for Val in this variant, amino acid 310 in table 2)

nucleotide 1077 is “T” in this variant compared to “C” in the Ellison et al sequence (silent mutation)

nucleotide 1197 is “G” in this variant compared to “A” in the Ellison et al sequence (silent mutation)

The fusion protein was expressed transiently in Cos1 cells using the pCDN vector system, as described in International Patent Application Publication number WO 96/04388. The mature protein was exported from the cells into the culture medium and was detected by anti-leptin antibody. It was shown to to have a size consistent with the predicted structure by Western blotting analysis under both reducing and nonreducing conditions.

TABLE 7
DNA sequence of ob/IgG1′GT′ chimera, 1203bp

```
ATGCATTGGGGAACCCTGTGCGGATTCTTGTGGCTTTGGCCCTATCTTTTCTATGTCCAA
                                                                                                                    60
GCTGTGCCCATCCAAAAAGTCCAAGATGACACCAAAACCCTCATCAAGACAATTGTCACC
                                                                                                                  120
AGGATCAATGACATTTCACACACGCAGTCAGTCTCCTCCAAACAGAAAGTCACCGGTTTG
                                                                                                                  180
GACTTCATTCCTGGGCTCCACCCCATCCTGACCCTGTCCAAGATGGACCAGACACTGGCA
                                                                                                                  240
GTCTACCAACAGATCCTCACATCGATGCCTTCCAGAAACGTGATCCAAATATCCAACGAC
                                                                                                                  300
CTGGAGAACCTCCGGGATCTTCTTCACGTGCTGGCCTTCTCTAAGAGCTGCCACTTGCCC
                                                                                                                  360
TGGGGCAGTGGCCTGGAGACCTTGGACAGCCTGCGGGGTGTCCTCGAGGCTTCAGGCTAC
                                                                                                                  420
TCCACAGAGGTGGTGGCCCTGAGCAGGCTGCAGGGGTCTCTGCAGGACATGCTCTGGCAG
                                                                                                                  480
CTGGACCTCAGCCCCGGGTGCGGTACCGAGCCCAAATCGGCCGACAAAACTCACACATGC
                                                                                                                  540
CCACCGTGCCCAGCACCTGAACTCCTGGGGGGACCGTCAGTCTTCCTCTTCCCCCCAAAA
                                                                                                                  600
CCCAAGGACACCCTCATGATCTCCCGGACCCCTGAGGTCACATGCGTGGTGGTGGACGTG
                                                                                                                  660
AGCCACGAAGACCCTGAGGTCAAGTTCAACTGGTACGTGGACGGCGTGGAGGTGCATAAT
                                                                                                                  720
GCCAAGACAAAGCCGCGGGAGGAGCAGTACAACAGCACGTACCGTGTGGTCAGCGTCCTC
                                                                                                                  780
ACCGTCCTGCACCAGGACTGGCTGAATGGCAAGGAGTACAAGTGCAAGGTCTCCAACAAA
                                                                                                                  840
GCCCTCCCAGCCCCCATCGAGAAAACCATCTCCAAAGCCAAAGGGCAGCCCCGAGAACCA
                                                                                                                  900
CAGGTGTACACCCTGCCCCCATCCCGGGAGGAGATGACCAAGAACCAGGTCAGCCTGACC
                                                                                                                  960
TCCCTGGTCAAAGGCTTCTATCCCAGCGACATCGCCGTGGAGTGGGAGAGCAATGGGCAG
                                                                                                                1020
CCGGAGAACAACTACAAGACCACGCCTCCCGTCCTGGACTCCGACGGCTCCTTCTTCCTC
                                                                                                                1080
TATAGCAAGCTCACCGTGGACAAGAGCAGGTGGCAGCAGGGGAACGTCTTCTCATGCTCC
                                                                                                                1140
GTGATGCATGAGGCTCTGCACAACCACTACACGCAGAAGAGCCTCTCCCTGTCTCCGGGT
                                                                                                                1200
AAG
1203
```

TABLE 8
Amino acid sequence of leptin/IgG1′GT′ chimera, 401aa

1 MHWGTLCGFL WLWPYLFYVQ AVPIQKVQDD TKTLIKTIVT
RINDISHTQS
51 VSSKQKVTGL DFIPGLHPIL TLSKMDQTLA VYQQILTSMP
SRNVIQISND
101 LENLRDLLHV LAFSKSCHLP WASGLETLDS LGGVLEASGY
STEVVALSRL
151 QGSLQDMLWQ LDLSPGCGTE PKSADKTHTC PPCPAPELLG
GPSVFLFPPK
201 PKDTLMISRT PEVTCVVVDV SHEDPEVKFN WYVDGVEVHN
AKTKPREEQY
251 NSTYRVVSVL TVLHQDWLNG KEYKCKVSNK ALPAPIEKTI
SKAKGQPREP
301 QVYTLPPSRE EMTKNQVSLT CLVKGFYPSD IAVEWBSNGQ
PENNYKTFPP
351 VLDSDGSFFL YSKLTVDKSR WQQGNVFSCS VMHEALHNHY
TQKSLSLSPG
401 K

1. Chimeric leptin or a chimeric mutant or derivative of leptin.

2. A chimera according to claim 1, wherein the leptin is human leptin.

3. A chimera according to claim 1 or claim 2, wherein the leptin or a mutant or variant thereof is fused to a human immunoglobulin domain or a mutant or variant thereof.

4. A chimera according to any one of claims 1 to 3, wherein the chimeric protein comprises one human immunoglobulin domain.

5. A chimera according to claim 4, wherein the human immunoglobulin domain is fused to the C-terminus of leptin.

6. A chimera according to any one of claims 1 to 4, which comprises a human immunoglobulin Fc domain.

7. A chimera according to claim 6, wherein the human immunoglobulin Fc domain is an IgG4PE variant, an IgG4, IgG1 or an IgG1GT variant, in particular the hinge-CH₂-CH₃ region in each case.

8. A chimera according to claim 7, wherein the variant a hinge-CH₂-CH₃ variant.

9. Chimeric leptin selected from the list consisting of:
leptin 1-167/IgG4 hinge-CH2-CH3;
leptin 1-167/IgG4 hinge-CH2-CH3 PE variant;
leptin 1-167/IgG1 hinge-CH2-CH3; and
leptin 1-167/IgG1 hinge-CH2-CH3 GT linker variant.

10. A process for preparing a chimera according to any one of claims 1 to 8, which process comprises expressing DNA encoding said compound in a recombinant host cell and recovering the product.

11. A process according to claim 10, which process comprises the steps of:
i) preparing a replicable expression vector capable, in a host cell, of expressing a DNA polymer comprising a nucleotide sequence that encodes said chimera;
ii) transforming a host cell with said vector;
iii) culturing said transformed host cell under conditions permitting expression of said DNA polymer to produce said chimera; and
iv) recovering said chimera.

12. A DNA polymer comprising a nucleotide sequence that encodes a chimera according to any one of claims 1 to 8.

13. A vector which comprises a DNA polymer according to claim 12.

14. A host cell transformed or transfected with a DNA polymer according to claim 12 or a vector according to claim 13.

15. A pharmaceutical composition comprising a chimera as claimed in claim 1 and a pharmaceutically acceptable carrier.

16. A chimera according to claim 1, for use as an active therapeutic substance.

17. A chimera according to claim 1, for use in the treatment of obesity or diseases associated with obesity.

18. A method for the treatment of obesity or diseases associated with in human or non-human mammal, which method comprises administering to the sufferer an effective, non-toxic amount of a chimera as claimed in claim 1.

19. A chimera as claimed in claim 1, for use in the cosmetic treatment of human or non-human mammals.

20. A method for the cosmetic treatment of a human or non-human mammal, which treatment comprises administering an effective, non-toxic amount of a compound of the invention to a human or non-human mammal in need thereof.


SEQUENCE LISTING (9 sequences)

SEQ ID NO: 1 - DNA, 1188 bp; single-stranded, linear
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccctgtccaagatggaccagacactggcagtctaccaacagatcctcacatcgatgccttccagaaacgtgatccaaatatccaacgacctggagaacctccgggatcttcttcacgtgctggccttctctaagagctgccacttgccctgggccagtggcctggagaccttggacagcctggggggtgtcctcgaggcttcaggctactccacagaggtggtggccctgagcaggctgcaggggtctctgcaggacatgctgtggcagctggacctcagccccgggtgcgagtccaaatatggtcccccatgcccatcatgcccagcacctgaatttctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 2 - protein, 396 aa
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - DNA, 984 bp; single-stranded, linear
gctagtaccaagggcccatccgtcttccccctggcgccctgctccaggagcacctccgagagcacggccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 4 - DNA, 1188 bp; single-stranded, linear
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccctgtccaagatggaccagacactggcagtctaccaacagatcctcacatcgatgccttccagaaacgtgatccaaatatccaacgacctggagaacctccgggatcttcttcacgtgctggccttctctaagagctgccacttgccctgggccagtggcctggagaccttggacagcctggggggtgtcctcgaggcttcaggctactccacagaggtggtggccctgagcaggctgcaggggtctctgcaggacatgctgtggcagctggacctcagccccgggtgcgagtccaaatatggtcccccatgcccaccatgcccagcgcctgaatttgaggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcatcgatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagttggagagcaatgggcagccggagaacaactacaagaccacgcctcccgtgctggactccgacggatccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 5 - protein, 396 aa
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - DNA, 1196 bp; single-stranded, linear
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccctgtccaagatggaccagacactggcagtctaccaacagatcctcacatcgatgccttccagaaacgtgatccaaatatccaacgacctggagaacctccgggatcttcttcacgtgctggccttctctaagagctgccacttgccctgggccagtggcctggagaccttggacagcctggggggtgtcctcgaggcttcaggctactccacagaggtggtggccctgagcaggctgcaggggtctctgcaggacatgctgtggcagctggacctcagccccgggtgcgagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgccggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaag

SEQ ID NO: 7 - protein, 399 aa
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1203 bp; single-stranded, linear
atgcattggggaaccctgtgcggattcttgtggctttggccctatcttttctatgtccaagctgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccctgtccaagatggaccagacactggcagtctaccaacagatcctcacatcgatgccttccagaaacgtgatccaaatatccaacgacctggagaacctccgggatcttcttcacgtgctggccttctctaagagctgccacttgccctgggccagtggcctggagaccttggacagcctggggggtgtcctcgaggcttcaggctactccacagaggtggtggccctgagcaggctgcaggggtctctgcaggacatgctgtggcagctggacctcagccccgggtgcggtaccgagcccaaatcggccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaag

SEQ ID NO: 9 - protein, 401 aa
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCGTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK